(19) United States
(12) Patent Application Publication
(10) Pub. No.: US 2010/0068155 A1
(43) Pub. Date: Mar. 18, 2010

(54) Reactant Formulations and Methods for Controlled Heating

(75) Inventors: Mingzu Lei, San Jose, CA (US); C.V. Krishnamohan Sharma, Santa Clara, CA (US); Hoi Sze Lau, Palo Alto, CA (US); Karen Wang, Cupertino, CA (US)

Correspondence Address: SWANSON & BRATSCHUN, L.L.C, 8210 SOUTHPARK TERRACE, LITTLETON, CO, 80120, US

(73) Assignee: ALEXZA PHARMACEUTICALS, INC., Mountain View, CA (US)

(21) Appl. No.: 12/211,628

(22) Filed: Sep. 16, 2008

(51) Int. Cl. A61K 9/72

(52) U.S. Cl. 424/40

(57) ABSTRACT

Disclosed herein are reactant formulations capable of undergoing an exothermic chemical reaction. The reactant formulations comprise a metal reducing agent, a metal-containing oxidizing agent, and manganese oxide in an amount effective to control a temperature profile of the reactant formulation. The manganese oxide may be, for example and without limitation, MnO, MnO₂, or Mn₃O₄. The manganese oxide may be present at a concentration within the range of 2 to 60 percent, and in other embodiments, 8 to 40 percent, by weight of the reactant formulation. Also disclosed are an article for vaporization of a vaporizable compound and a heating unit.

### TECHNICAL FIELD

The present invention pertains to chemical reactant formulations with heat buffering properties and/or controllable heating properties. Such chemical reactant formulations can be employed in a variety of applications, for example, in aerosol drug delivery devices for the delivery of therapeutically effective agents by inhalation.

### BACKGROUND

Pulmonary delivery is recognized as an effective means of administering physiologically active compounds to a patient for the treatment of diseases and disorders. Devices developed for pulmonary delivery typically provide an aerosol of a physiologically active compound that can be inhaled by a patient. The inhaled compound can be used to treat conditions in a patient's respiratory tract and/or enter the patient's systemic circulation to treat conditions in other areas of the body. Devices for generating aerosols of physiologically active compounds include nebulizers, pressurized metered-dose inhalers, and dry powder inhalers. Nebulizers generate an aerosol through atomization of liquid drug solutions, while pressurized metered-dose inhalers and dry powder inhalers are based on suspension and dispersion of dry powder in an airflow.

Aerosols containing physiologically active compounds can be formed by vaporizing a substance in an airflow to produce a condensation aerosol comprising the active compounds. A condensation aerosol is formed, for example, when a gas phase substance condenses to form particles. Examples of devices and methods employing vaporization methods to produce condensation aerosols are disclosed in U.S. Pat. No. 7,090,830, entitled “Drug Condensation Aerosols and Kits”, issued Aug. 15, 2006, and U.S. application Ser. No. 10/850,895, filed May 20, 2004 and published as US-2005-0079166 on April 14, 2005, entitled “Self-Contained Heating Unit and Drug-Supply Unit Employing Same”, each of which is incorporated herein by reference in its entirety.

A condensation aerosol comprising a drug may be produced by rapidly heating and vaporizing a thin layer of a drug coated onto a substrate such that there is minimal degradation of the drug. The vaporized drug then condenses to produce an aerosol characterized by high purity. For use in portable, hand-held medical devices, the heat source for vaporizing the drug is preferably compact and capable of producing a rapid heat impulse. A variety of heat sources for such devices are summarized below.

Chemically based heating units typically include a chemical reactant formulation (also referred to herein as a “fuel”) which is capable of undergoing an exothermic metal oxidation-reduction reaction within an enclosure (see, e.g., U.S. application Ser. No. 10/850,895, published as US-2005-0079166 on Apr. 14, 2005). The chemical reactant formulation is typically coated as a layer onto the surface of a substrate. Once a portion of the fuel is ignited, the heat generated by the oxidation-reduction reaction can ignite adjacent unburned fuel until all of the fuel is consumed in the process of the chemical reaction. The fuel is typically ignited by the application of energy to at least a portion of the fuel. Energy absorbed by the fuel, or by an element in contact with the fuel, heats the fuel to a temperature above the auto-ignition temperature of the reactants (i.e., the minimum temperature required to initiate or cause self-sustaining combustion in the absence of a combustion source or flame). However, the peak temperature that is reached by the substrate upon ignition of the fuel may be affected by the coating density of reactant formulation on the substrate surface (i.e., greater coating densities tend to lead to increased peak temperatures).

Other approaches have also been employed for providing heat to a drug delivery device, for example, using electrochemical interactions, where compounds that interact electrochemically after initiation in an exothermic reaction are used to generate heat. Exothermic electrochemical reactions include reactions of a metallic agent and an electrolyte, such as a mixture of magnesium granules and iron particles as the metallic agent, and granular potassium chloride crystals as the electrolyte. In the presence of water, heat is generated by the exothermic hydroxylation of magnesium, where the rate of hydroxylation is accelerated in a controlled manner by the electrochemical interaction between magnesium and iron, which is initiated when the potassium chloride electrolyte dissociates upon contact with the liquid water. Electrochemical interactions have been used, for example, in the smoking industry to volatilize tobacco for inhalation (U.S. Pat. Nos. 5,285,798; 4,941,483; 5,593,792).

As mentioned above, previously known chemical reactant formulations can be sensitive to the amount of reactant formulation that is coated onto the surface of a substrate. Furthermore, it can be difficult to provide control over the maximum temperature (Tₘₐₓ) reached by the substrate using prior art chemical reactant formulations.

The general correlation between stainless steel foil substrate temperature and reactant coating mass is illustrated in FIG. 1, which is a graph 100 showing peak substrate surface temperature 102 achieved by foil substrates as a function of reactant coating mass 104. For example, a relatively small variation in reactant coating mass can result in a large variation in peak substrate surface temperature. In such a scenario, reactant coating thickness uniformity must be tightly controlled to avoid wide fluctuations in peak temperature across the surface of the substrate.

As can be seen from graph 100, the smaller the slope of the peak substrate surface temperature vs. reactant coating mass curve, the better the “heat buffering” properties of the reactant coating formulation. The term “heat buffering zone” refers to a range of reactant formulation coating densities wherein the peak substrate surface temperature that is attained upon ignition of the reactant formulation is not significantly affected by either an increase or decrease in reactant formulation coating mass per surface area. The “ideal” reactant coating formulation would therefore have a flat slope (indicated by the dotted line on graph 100), such that the peak substrate surface temperature does not increase at all with increased reactant formulation coating density. In such a scenario, reactant coating thickness uniformity need not be so tightly controlled, providing increased ease of manufacturing. Such a reactant coating formulation could significantly decrease aerosol drug delivery device manufacturing costs (e.g., by eliminating steps in the manufacturing process, such as weighing each device after production) and improve product yield, as well as improve the reliability of device performance.

Therefore, there remains a need in the art for chemical reactant formulations that provide for control over substrate temperature during the heating process.

### SUMMARY

We have developed novel chemical reactant formulations that exhibit heat buffering properties. In particular, we have discovered that the heat output from such chemical reactant formulations is less sensitive than previously known chemical reactant formulations to the reactant coating density on the surface of a substrate (such as a stainless steel foil). More importantly, these new reactant formulations predetermine the maximum temperature that the substrate surface can attain for a given reactant coating density.

Zr:Fe₂O₃: Laponite-based aqueous fuel formulations form stable slurry dispersions and provide 300-500° C. surface temperatures on 3-5 mil thick stainless steel foils. We have discovered that when the oxidizer manganese oxide (for example in the form of MnO₂) is added to the Zr:Fe₂O₃: Laponite formulations in certain percentages, the reactant formulations exhibit surprising heat buffering properties, such that peak substrate temperatures do not increase with increasing fuel coating weights. Instead, the steel foils are heated for a longer period of time. The presence of the manganese oxide in the reactant formulation enhances the exothermic reaction propagation speed, making it possible to achieve faster, more controllable heating rates.

Reactant formulations with heat buffering properties offer numerous advantages over prior art reactant formulations. For example, the heat buffering reactant formulations disclosed herein are capable of allowing the peak substrate surface temperature (Tₘₐₓ) to be selected such that a desired temperature is reached, but not significantly exceeded within a given reactant coating density range. Alternatively, the heat buffering reactant formulations of the invention can be tailored such that Tₘₐₓ falls within a particular temperature range. As such, the reactant formulations of the invention provide tighter temperature control and highly reliable/predictable drug purities, dosages, and particle sizes than prior art reactant formulations. Furthermore, use of the present reactant formulations to vaporize drugs may potentially improve the purity of the aerosolized drug. As a result of the improved peak temperature control (i.e., no accidental higher temperatures) and decreased levels of impurities, the reactant formulations of the invention may also provide improved aerosol drug delivery device safety.

The reactant formulations of the invention are less sensitive to variations in reactant coating density than previously known chemical reactant formulations. Due to tighter control over the manufacturing process resulting in higher device yields, manufacturing costs of the reactant formulations may be reduced. Furthermore, because the amount of expensive metal reactant materials (e.g., zirconium) in the reactant formulations is reduced, starting material costs are lower.

Accordingly, disclosed herein are reactant formulations capable of undergoing an exothermic chemical reaction. According to the present invention, the reactant formulations comprise a metal reducing agent, a metal-containing oxidizing agent, and manganese oxide in an amount effective to modulate the heat output of the reactant formulation. The manganese oxide may be, for example and without limitation, MnO, MnO₂, or Mn₃O₄, with MnO₂ in particular yielding good results. In some embodiments the manganese oxide is present at a concentration within the range of 2 to 60 percent; in other embodiments 8 to 40 percent; in further embodiments 20 to 40 percent by weight of the reactant formulation.

The metal reducing agent may be selected from the group consisting of zirconium, molybdenum, magnesium, calcium, strontium, barium, boron, titanium, vanadium, niobium, tantalum, chromium, tungsten, manganese, iron, cobalt, nickel, copper, zinc, cadmium, tin, antimony, bismuth, aluminum, and silicon, for example and without limitation. In one embodiment the metal reducing agent is zirconium, which may be present at a concentration within the range of 30 to 90 percent; alternatively 40 to 80 percent; and further alternatively, 40 to 70 percent by weight of the reactant formulation.

The metal-containing oxidizing agent may be selected from the group consisting of transition metal oxides, lanthanide metal oxides, and mixed metal oxides. In some embodiments, the metal-containing oxidizing agent is a transition metal oxide selected from the group consisting of oxides of iron, copper, cobalt, molybdenum, vanadium, chromium, manganese, silver, tungsten, magnesium, and niobium, for example and without limitation. In one embodiment, the metal-containing oxidizing agent is iron oxide, which may be present at a concentration within the range of 5 to 40 percent; alternatively 10 to 30 percent; and further alternatively, 15 to 30 percent by weight of the reactant formulation.

Reactant formulations according to the present invention include, for example and without limitation, Zr:Fe₂O₃:MnO₂, Zr:CuO:MnO₂, Zr:Co₃O₄:MnO₂, Zr:Co₂O₃:MnO₂, and Zr:MoO₃:MnO₂.

The reactant formulation may further comprise a binding agent, which may be selected from the group consisting of clays, metal silicates, phosphate-containing materials, alkoxides, metal oxides, inorganic polyanions, inorganic polycations, inorganic sol-gel materials, synthetic ion exchange resins, zeolites, and diatomaceous earth.

Also disclosed herein is an article useful for vaporization of a vaporizable compound. The article comprises a substrate having a first surface and a second surface. At least a portion of the first surface is coated with a reactant formulation capable of undergoing an exothermic chemical reaction. The reactant formulation comprises a metal reducing agent, a metal oxidizing agent, and manganese oxide in an amount effective to modulate the heat output of the reactant formulation. At least a portion of the second surface of the substrate is coated with a vaporizable compound, typically, a drug.

Heating units which utilize the chemical reactant formulation of the invention are also contemplated herein. Such heating units comprise a substrate having a first surface and a second surface, where a reactant formulation capable of undergoing an exothermic chemical reaction is disposed upon at least a portion of the first surface of the substrate. The chemical reactant formulation comprises a metal reducing agent, a metal-containing oxidizing agent, and manganese oxide in an amount effective to modulate the heat output of the reactant formulation upon initiation of the reactant formulation. The heating unit may also further comprise at least one vaporizable compound disposed upon at least a portion of the second surface of the substrate, where the vaporizable compound is typically a drug.

Also disclosed herein is a method of modulating the heat output of a chemical reactant formulation. The method comprises combining the chemical reactant formulation with manganese oxide, wherein the manganese oxide is present at a concentration within the range of 2 to 60 percent by weight of the chemical reactant formulation. The percentage of manganese oxide within the chemical reactant formulation is selected so as to control the temperature profile of the chemical reactant formulation upon initiation of the chemical reactant formulation. By “control the temperature profile” we mean to reduce the slope of a plot of temperature versus reactant mass (see FIG. 1), alter the heating rate, vary the wavefront speed of the initiated chemical reactant formulation propagating along the substrate (see FIG. 3), change the maximum temperature that can be attained by the initiated chemical reactant formulation(see FIG. 2), and/or affect the time period over which the initiated chemical reactant formulation is able to maintain a desired temperature.

Also disclosed herein is a method of improving the uniformity of heating upon ignition of a chemical reactant formulation. The method comprises combining the chemical reactant formulation with manganese oxide, wherein the manganese oxide is present at a concentration within the range of 2 to 60 weight percent of the chemical reactant formulation. Uniformity of heating upon initiation of the chemical reactant formulation is controlled by varying the percentage of manganese oxide within the chemical reactant formulation.

Also disclosed herein is a method of improving the adhesion of a chemical reactant formulation to a substrate upon ignition of the chemical reactant formulation. The method comprises combining the chemical reactant formulation with manganese oxide, wherein the manganese oxide is present at a concentration within the range of 2 to 60 weight percent of the chemical reactant formulation. The degree to which the chemical reactant formulation adheres to substrate may be enhanced by varying the percentage of manganese oxide within the chemical reactant formulation.

Also disclosed herein is a method of modulating the maximum temperature (Tₘₐₓ) achieved upon initiation of a chemical reactant formulation. The method comprises combining the chemical reactant formulation with manganese oxide, wherein the manganese oxide is present at an amount within the range of 2 to 60 weight percent of the chemical reactant formulation.

### BRIEF DESCRIPTION OF THE DRAWINGS

FIG. 1 is a schematic graph showing peak substrate surface temperature achieved by a stainless steel foil substrate as a function of chemical reactant formulation coating mass.

FIG. 2 is a graph showing peak substrate surface temperature achieved by a stainless steel foil substrate as a function of chemical reactant formulation coating density for three different chemical reactant formulations

FIGS. 3A-3B are schematic representations of infrared thermal images of stainless steel foil substrates heated using self-propagating chemical reactions.

FIG. 3C is a schematic representation of infrared thermal images of a stainless steel foil substrate heated electrically through resistive heating.

FIG. 4 is a graph showing the effect of reactant propagation speed on aerosol particle size upon vaporization for two different drugs, prochlorperazine and bumetanide.

FIG. 5 is a cross-sectional side view of an embodiment of a heating unit wherein a first substrate and a second substrate are part of a single component folded together and sealed to form a unitary structure containing chemical reactant material within.

FIG. 6 is a schematic cross-sectional side view of an embodiment of a drug supply unit which includes a heating unit which further includes a chemical reactant material, an igniter, and a thin drug layer.

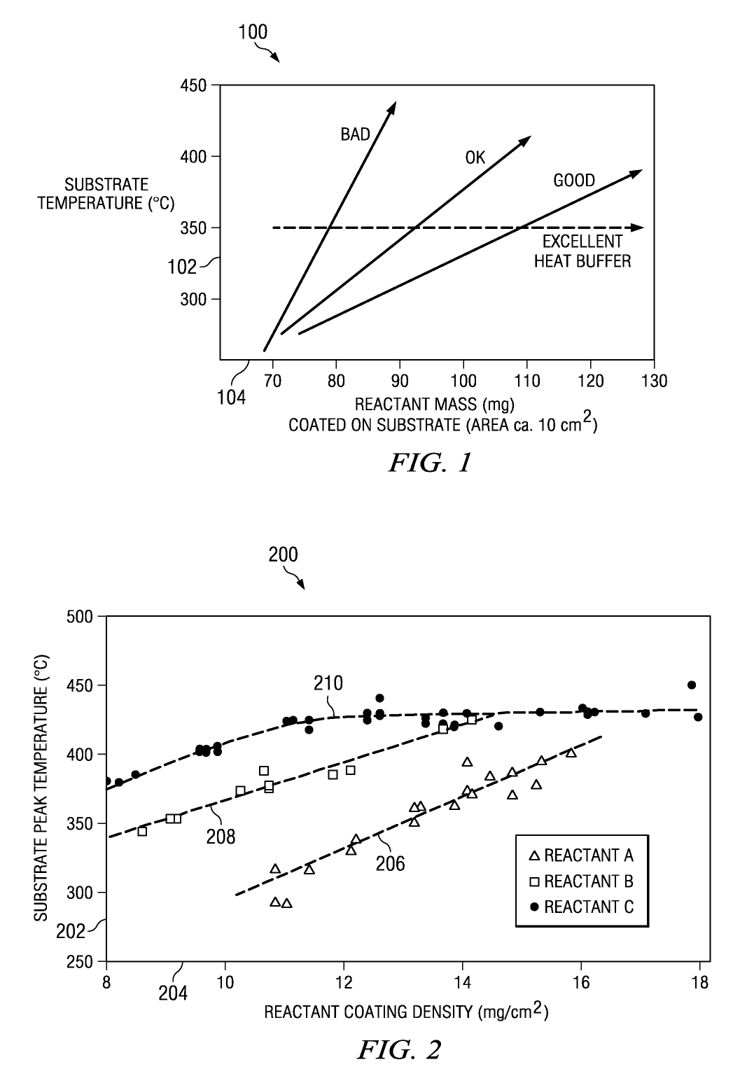

### DETAILED DESCRIPTION

Descriptions and examples of each of the components of the reactant coating formulations of the invention, and of heating units employing such reactant coating formulations, are provided below.

### Heat-Buffering Reactant Coating Formulations

Disclosed herein are reactant formulations capable of undergoing an exothermic chemical reaction. Such chemical reactant formulations can be prepared in many forms such as, for example and not by way of limitation, solids, gels, liquids, and combinations thereof. Such chemical reactant formulations can produce heat by means of an exothermic chemical reaction including, for example, a metal oxidation-reduction reaction.

An oxidation-reduction reaction refers to a chemical reaction in which one compound gains electrons and another compound loses electrons. The compound that gains electrons is referred to as an oxidizing agent, and the compound that loses electrons is referred to as a reducing agent. An example of an oxidation-reduction reaction is a chemical reaction of a compound with molecular oxygen (O₂) or with an oxygen-containing compound that adds one or more oxygen atoms to the compound being oxidized. During the oxidation-reduction reaction, the molecular oxygen or the oxygen-containing compound is reduced by the compound being oxidized. The compound providing oxygen acts as the oxidizer or oxidizing agent. The compound being oxidized acts as the reducing agent. Oxidation-reduction reactions can be exothermic, meaning that the reactions generate heat. An example of an exothermic oxidation-reduction reaction is the thermite reaction of a metal with a metal-containing oxidizing agent.

The chemical reactant formulations of the present invention comprise a metal reducing agent, a metal-containing oxidizing agent, and manganese oxide in an amount effective to modulate the heat output of the reactant formulation. The manganese oxide may be, for example and without limitation, MnO, MnO₂, or Mn₃O₄, with MnO₂ yielding good results. In some embodiments the manganese oxide is present at a concentration within the range of 2 to 60 percent; in other embodiments 8 to 40 percent; in further embodiments within the range of 20 to 40 percent, by weight of the reactant formulation.

The metal reducing agent may be selected from the group consisting of zirconium, molybdenum, magnesium, calcium, strontium, barium, boron, titanium, vanadium, niobium, tantalum, chromium, tungsten, manganese, iron, cobalt, nickel, copper, zinc, cadmium, tin, antimony, bismuth, aluminum, and silicon, for example and without limitation. In one embodiment the metal reducing agent is zirconium, which may be present at a concentration within the range of 30 to 90 percent; alternatively 40 to 80 percent; in further embodiments 40 to 70 percent by weight of the reactant formulation. In certain embodiments, the metal reducing agent can comprise more than one metal reducing agent.

The metal-containing oxidizing agent may be selected from the group consisting of transition metal oxides, lanthanide metal oxides, and mixed metal oxides. In some embodiments, the metal-containing oxidizing agent is a transition metal oxide selected from the group consisting of oxides of iron (e.g., Fe₂O₃), copper (e.g., CuO), cobalt (e.g., CO₃O₄), molybdenum (e.g., MoO₃), vanadium (e.g., V₂O₅), chromium (e.g., CrO₃, Cr₂O₃), manganese (e.g., MnO₂), silver (e.g., Ag₂O), tungsten (e.g., WO₃), (e.g., MgO), and niobium (e.g., Nb₂O₅), for example and without limitation. In certain embodiments, the metal-containing oxidizing agent can include more than one metal-containing oxidizing agent.

In some embodiments, the metal-containing oxidizing agent is iron oxide, which is present at a concentration within the range of 5 to 40 percent; alternatively 10 to 30 percent; and further alternatively, 15 to 30 percent by weight of the reactant formulation.

Preferred reactant formulations according to the present invention include, for example and without limitation, Zr:Fe₂O₃:MnO₂, Zr:CuO:MnO₂, Zr:Co₃O₄:MnO₂, Zr:Co₂O₃:MnO₂, and Zr:MoO₃:MnO₂.

The ratio of metal reducing agent to metal-containing oxidizing agent affects the ignition temperature and the burn characteristics of the reactant coating formulation. In certain embodiments, the amount of oxidizing agent in the reactant formulation can be related to the molar amount of the oxidizers at or near the eutectic point for the reactant formulation. In certain embodiments, the oxidizing agent can be the major component and, in others, the metal reducing agent can be the major component. Those of skill in the art are able to determine the appropriate amount of each component based on the stoichiometry of the chemical reaction and/or by routine experimentation. Also, the particle size of the metal reducing agent and the metal-containing oxidizing agent can be varied to determine the burn rate, with smaller particle sizes selected for a faster burn (see, for example, U.S. Pat. No. 5,603,350).

The reactant formulation may further comprise a binding agent. A binding agent refers to an additive that produces bonding strength in a final product. The binding agent can impart bonding strength, for example, by forming a bridge, film, matrix, and/or chemically self-reacting and/or reacting with other constituents of the formulation, preferably imparting added resistance to cracking within the film.

Examples of binding agents which are useful in the present invention include, but are not limited to, clays, metal silicates (including soluble silicates such as sodium, potassium, and aluminum silicates), phosphate-containing metals (in particular, phosphates of metals such as copper, zinc, iron, aluminum, manganese, and titanium), alkoxides, metal oxides, inorganic polyanions, inorganic polycations, and inorganic sol-gel materials, such as alumina or silica-based sols. Binding agents can also comprise materials such as synthetic ion exchange resins, zeolites (synthetic or naturally occurring), and/or diatomaceous earth.

Specific examples of binding agents useful in the chemical reactant formulations of the present invention include, but are not limited to, clays such as Laponite® or Cloisite® additives (manufactured by Rockwood Additives Limited, Widnes, United Kingdom, and available from Southern Clay Products, Inc., Gonzales, Tex.); montmorillonite (a very soft phyllosilicate mineral that typically forms microscopic crystals); metal alkoxides, such as those represented by the formula R—Si(OR)ₙ and M(OR)ₙ, where n can be 3 or 4, and M can be Ti, Zr, Al, B, or another metal; and colloidal particles based on transition metal hydroxides or oxides.

In one embodiment, the reactant formulation includes a Laponite® additive. Laponite additives are synthetic layered silicates, in particular, magnesium phyllosilicates, with a structure resembling that of the natural clay mineral hectorite (Na_{0.4}Mg_{2.7}Li_{0.3}SiO₁₀(OH)₂). Laponite RD (59.5% SiO₂: 27.5% MgO: 0.8% Li₂O: 2.8% Na₂O) is a commercial grade material which, when added to water, rapidly disperses to form a gel. Laponite RDS (54.5% SiO2: 26% MgO: 0.8% Li₂O: 5.6% Na₂O: 4.1% P₂O₅) is a commercially available sol-forming grade of Laponite modified with a polyphosphate dispersing agent, or peptizer, to delay rheological activity until the Laponite RDS is added as a dispersion into a formulation. A sol refers to a colloid having a continuous liquid phase in which solid is suspended in a liquid. In the presence of electrolytes, Laponite additives can act as gelling, binding, and/or thixotropic agents. Thixotropy refers to the property of a material to exhibit decreased viscosity under shear.

Minimizing the reactant coating thickness can facilitate control of the heating process, as well as facilitate miniaturization of a drug supply unit incorporating a heating unit of the invention. The reactant coating formulation can be disposed on the substrate as a film or layer having a thickness within the range of 10 μm to 500 μm; alternatively within the range of 10 μm to 100 μm; and further alternatively, within the range of 20 μm to 60 μm.

It is advantageous that the reactant formulations disclosed herein adhere to the surface of the substrate, and that the constituents of the reactant formulation adhere to each other and maintain physical integrity. In addition, physical inspection has shown that the reactant formulations remain adhered to the substrate surface and maintain physical integrity during processing and storage, during which time the reactant coating may be exposed to a variety of mechanical and environmental conditions.

The reactant formulation can be any appropriate shape and have any appropriate dimensions. For example, the reactant formulation can be shaped for insertion into a square or rectangular heating unit. To increase the contact/binding area between the substrate surface and the overlying adhesive layer, and thereby enhance the rigidity of the adhesive layer during or after ignition, a slurry comprising the reactant formulation can be printed as lines or patches on a substrate surface.

### Substrates and Heating Units

A variety of substrates are contemplated for use in the preparation of heating units which are coated with the heat-buffering reactant formulation of the invention. Examples of materials include metals, metal alloys, and ceramics (including glasses).

Presently preferred substrates are thin to facilitate heat transfer from the interior to the exterior surface and/or to minimize the thermal mass of the device. In certain embodiments, the substrate has a thickness in the range of 0.001 inch to 0.020 inch; in other embodiments, in the range of 0.001 inch to 0.010 inch; in the range of 0.002 inch to 0.006 inch; or in the range of 0.002 inch to 0.005 inch.

In certain embodiments, thinner substrates can facilitate more rapid and more homogeneous heating of the exterior surface with a lesser amount of fuel material compared to a thicker substrate. The substrate can also provide structural support for the fuel material and an optional material to be heated, such as for example, a drug film.

One presently preferred substrate is a metal foil. Examples of metal foils include stainless steel, copper, aluminum, and nickel, as well as alloys thereof.

Alternatively, the substrate may comprise a ceramic. As used herein, the term “ceramic” refers to complex compounds and solid solutions of both metallic and nonmetallic elements joined by ionic and covalent bonds. Most often, ceramic materials are a combination of inorganic elements, although they may occasionally contain carbon. Examples of ceramic materials include, but are not limited to, metallic oxides (such as oxides of aluminum, silicon, magnesium, zirconium, titanium, chromium, lanthanum, hafnium, yttrium, and mixtures thereof) and non-oxide compounds including, but not limited to, carbides (such as carbides of titanium, tungsten, boron, silicon, and mixtures thereof), silicides (such as molybdenum disicilicide), nitrides (such as nitrides of boron, aluminum, titanium, silicon, and mixtures thereof) and borides (such as borides of tungsten, titanium, and mixtures thereof), and mixtures thereof; spinels, titanates (such as barium titanate, strontium titanate, iron titanate), ceramic super conductors, zeolites, ceramic solid ionic conductors (such as yittria-stabilized zirconia, beta-alumina, and cerates).

Heating units which are coated with the reactant formulation of the present invention may also further comprise a second substrate having a first surface and a second surface. The second substrate may be configured with respect to the first substrate to provide a “sandwich”-like structure. In such a scenario, the heating unit comprises a first substrate having a first surface and a second surface, a reactant formulation according to the present invention disposed upon a portion of the first surface of the first substrate, at least one adhesive layer disposed upon at least a portion of the fuel material and/or the substrate, and a second substrate having a first and second surface disposed opposite the first surface of the first substrate.

Alternatively, heating units which are coated with the reactant formulation of the invention can be configured such that the first and second substrates are part of a single component which can be folded to form a unitary structure having the chemical reactant material contained within. Upon folding the first and second substrate materials together, they can be sealed (for example, by use of adhesive, crimping, or welding) so as to form a highly stable heating device. FIG. 5 is a cross-sectional side view of such a heating unit 500. A chemical reactant formulation 506 is coated on the stainless steel substrate 502, an igniter 504 is located in operative proximity to the reactant formulation, and the substrate is folded together and sealed by seamwelding.

One of the many advantages of such heating units is the adjustability of surface area size for the application of one or more vaporizable compounds (or multiple doses of the same vaporizable compound) thereto. Embodiments of heating units can be prepared from substrates having surface areas of at least 0.2 cm², with other embodiments having surface areas within the range of 0.2 cm² to 50 cm² per heating unit. As used herein, the term “surface area per heating unit” refers to the surface area associated with a single source of the fuel material. As used herein, the term “surface area per substrate” refers to the total surface area associated with all sources of fuel material on a single substrate. For purposes herein, a heating unit may include multiple sources of reactant formulation. As used herein, the term “surface area per heating device” refers to the total surface area associated with all sources of reactant formulation in a heating unit, which may include multiple substrates.

Another advantage of such heating units is their relatively small dimensions. The heating units can be prepared to have a thickness of 10 mm or less, with thicknesses as low as 0.04 mm being possible. The thinness of the heating units allowed multiple units to be stacked on top of each other to increase the heated surface area or to deliver multiple doses from a smaller inhalation drug delivery device.

### Igniters

Such heating units further comprise at least one igniter to facilitate ignition of the reactant formulation. Also contemplated herein are heating units comprising a plurality of igniters. The plurality of igniters helps to ensure complete ignition of all of the reactant formulation. In one embodiment of the heating units featuring multiple igniters, a plurality of igniters are attached to a single coating of reactant formulation. In another embodiment, there are multiple coatings of reactant formulation, each having at least one igniter.

The igniter can comprise any device that is capable of igniting the reactant formulation to generate a self-sustaining oxidation-reduction reaction. A variety of devices and methods can be used for this purpose, for example and without limitation, optical igniters, percussive igniters, and electrical igniters, as described, for example, in U.S. Patent Publication Nos. 2005/0079166; 2004/0234914; and 2004/0234916.

Alternatively, the igniter can be a printable igniter of the type described in commonly assigned, copending U.S. patent application Ser. No. 12/211,554 (Attorney Docket No. 84.01R), filed on even date herewith. Such an igniter comprises at least two conductors in a spaced-apart configuration, and a conductive layer bridging the at least two conductors. The conductive layer, which is adapted to initiate and produce a “glow” (i. e., localized heat) upon application of electrical power, has an electrical resistance that is greater than the electrical resistance of both of the at least two conductors. Upon initiation of the conductive layer, heat from the exothermic oxidation of the conductive layer composition is generated sufficient to actuate the reactant formulation.

Once a portion of the reactant formulation is ignited, the heat generated by the oxidation-reduction reaction can ignite adjacent unburnt fuel until all of the fuel is consumed. The exothermic oxidation-reduction reaction can be initiated by the application of energy to at least a portion of the reactant formulation. Energy absorbed by the reactant formulation or by an element in contact with the reactant formulation can be converted to heat. When the reactant formulation is heated to a temperature above the auto-ignition temperature of the reactants, the oxidation-reduction reaction will initiate, igniting the reactant formulation in a self-sustaining reaction until the fuel is consumed.

The auto-ignition temperature of a reactant formulation comprising a metal reducing agent and a metal-containing oxidizing agent as disclosed herein may be in the range of 200° C. to 800° C. In certain embodiments, the auto-ignition temperature is in the range of 300° C. to 700° C.

Energy can be applied to ignite the reactant formulation using a number of methods. For example, a resistive heating element can be positioned in thermal contact with the reactant formulation which, when a current is applied, can heat the reactant formulation to its auto-ignition temperature. An electromagnetic radiation source can be directed at the reactant formulation which, when absorbed, can heat the reactant formulation to its auto-ignition temperature. An electromagnetic source can include, for example and not by way of limitation, lasers, diodes, flashlamps, and microwave sources.

Inductive heating can heat the reactant formulation by applying an alternating magnetic field that can be absorbed by materials having high magnetic permeability, either within the reactant formulation or in thermal contact with the reactant formulation. The source of energy can be focused onto the absorbing material to increase the energy density to produce a higher local temperature and thereby facilitate ignition. In certain embodiments, the reactant formulation can be ignited by percussive forces.

As is known in the art, for example, in the pyrotechnic industry, sparks can be used to safely and efficiently ignite chemical reactant formulations. Sparks refer to an electrical breakdown of a dielectric medium or the ejection of burning particles. In the first sense, an electrical breakdown can be produced, for example, between separated electrodes to which a voltage is applied. Sparks can also be produced by ionizing compounds in an intense laser radiation field. Examples of burning particles include those produced by friction and break sparks produced by intermittent electrical current. Sparks of sufficient energy incident on a chemical reactant formulation can initiate the self-sustaining oxidation-reduction reaction.

When sufficiently heated, the exothermic oxidation-reduction reaction of the reactant formulation can produce sparks, as well as radiation energy. Thus, in certain embodiments, reliable, reproducible, and controlled ignition of the reactant formulation can be facilitated by the use of an initiator composition capable of reacting in an exothermic oxidation-reduction reaction. Suitable initiator compositions are described, for example, in copending U.S. patent application Ser. Nos. 10/850,895 (filed May 20, 2004, now published as US-2005-0079166 on Apr. 14, 2005, and entitled “Self-Contained Heating Unit and Drug-Supply Unit Employing Same” and 10/851,018, now U.S. Pat. No. 7,402,777, (issued Jul. 22, 2008, and entitled “Multiple Dose Condensation Aerosol Devices and Methods of Forming Condensation Aerosols”) the disclosures of which are hereby incorporated by reference in their entireties.

Energy sufficient to heat the initiator composition to its auto-ignition temperature can be applied to the initiator composition and/or the support on which the initiator composition is disposed. The energy source can be any of those disclosed herein, such as resistive heating, radiative heating, inductive heating, optical heating, and percussive heating. In embodiments in which the initiator composition is capable of absorbing the incident energy, the support can comprise a thermally insulating material. In certain embodiments, the incident energy can be applied to a thermally conductive support that can heat the initiator composition above its auto-ignition temperature by thermal conduction.

In certain embodiments, the energy source can be an electrically resistive heating element. The electrically resistive heating element can comprise any material that can maintain integrity at the auto-ignition temperature of the initiator composition. In certain embodiments, the heating element can comprise an elemental metal such as tungsten, an alloy such as nichrome, or other material such as carbon. Materials suitable for resistive heating elements are known in the art. The resistive heating element can have any appropriate form. For example, the resistive heating element can be in the form of a wire, filament, ribbon, or foil. In certain embodiments, the electrical resistance of the heating unit can range from 2 Ω to 6 Ω. The appropriate resistivity of the heating element can at least in part be determined by the current of the power source, the desired auto ignition temperature, or the desired ignition time. In certain embodiments, the auto-ignition temperature of the initiator composition can range from 200° C. to 800° C. In other embodiments, the auto-ignition temperature of the initiator composition can range from 300° C. to 700° C. The resistive heating element can be electrically connected and suspended between two electrodes electrically connected to a power source.

Upon ignition of the reactant formulation, an exothermic oxidation-reduction reaction produces a considerable amount of energy in a short time, such as for example, in certain embodiments less than 1 second, in certain embodiments less than 500 milliseconds, and in certain embodiments less than 250 milliseconds. When used in enclosed heating units, by minimizing the quantity of reactants and the reaction conditions, the reaction can be controlled, but can result in a slow release of heat and/or a modest temperature rise. The temperature rise can exceed 200° C., and in some applications can exceed 250° C. or even 300° C. In certain applications, it can be useful to rapidly heat a substrate to temperatures in excess of 200° C. within 1 second or less. Such rapid intense thermal pulses can be useful for vaporizing pharmaceutical compositions to produce aerosols. A rapid intense thermal pulse can be produced using an exothermic oxidation-reduction reaction and, in particular, a thermite reaction involving a metal reducing agent and a metal-containing oxidizing agent.

The temperature to which one portion of the substrate is heated can be varied with respect to the temperature to which another portion of the substrate is heated in a variety of ways, thereby controlling the rate and/or time of delivery of one or more vaporizable compounds disposed upon at least a portion of the second surface of the substrate.

Thus, for example, in order to maximize the range of agents which can be heated employing heating units according to the present invention, the ratio of metal reducing agent to metal-containing oxidizing agent can be varied at different locations on the surface of the substrate, thereby providing different temperature maxima at different locations on the surface of the substrate upon ignition of the fuel material. This allows different areas on the surface of the substrate to be exposed to different temperatures, which allows the vaporization of drugs with different heating requirements, optionally at different times.

Similarly, the quantity of fuel material applied to the substrate can be varied at different locations on the first surface of the substrate, so as to achieve different temperature maxima upon ignition of the fuel material.

In any event, it is generally desirable to be able to rapidly heat a portion of the substrate to an elevated temperature (for example, a temperature of at least 200° C.) within, at most, 3 seconds following ignition of the fuel material. In other embodiments, heating of a portion of the substrate to an elevated temperature occurs within 2 seconds, or others within 1 second, and in others within 0.5 seconds.

### Drug Supply Units

Heating units according to the present invention may optionally further comprise at least one vaporizable compound disposed upon at least a portion of a second surface of the substrate to form a drug supply unit. When the heating unit comprises two substrates, the heating unit may further comprise at least one vaporizable compound disposed upon at least a portion of the second surface of the second substrate. Such a configuration allows for the delivery of two different vaporizable compounds at the same time, one from the outer surface of each substrate.

FIG. 6 is a schematic cross-sectional side view of an embodiment of such a drug supply unit 600. In the drug supply unit 600 a first substrate and a second substrate are part of a single component 602. A reactant formulation 606 is applied to a first surface of the component 602, an igniter 604 is located in operative proximity to the reactant formulation 606 and the component 602 is folded together and sealed by crimping or, seam welding, adhesives or other methods to hermetically seal the reactant 606 therein.

A wide variety of vaporizable compounds can be disposed on the heating devices of the invention and subsequently vaporized. Examples of vaporizable compounds include physiologically active compounds, industrially important compounds for which vaporization is desirable, and compounds which are useful for a variety of applications when converted into the vapor state, for example, air-freshening agents.

In accordance with one embodiment of the present invention, there are provided drug supply units comprising a heating unit as described herein, and at least one drug disposed on at least a portion of a second surface of the substrate.

A variety of drugs can be vaporized for delivery according to the present invention. As used herein, the term “drug” refers to any compound for therapeutic use or non-therapeutic use, including therapeutic agents and substances. As used herein, the term “therapeutic agent” refers to any compound suitable for use in the diagnosis, cure, mitigation, treatment, or prevention of a disease, and any compound used in the mitigation or treatment of symptoms of disease (where the term “substances” refers to compounds used for non-therapeutic uses, for example, for a recreational or experimental purpose).

Classes of drugs contemplated for use in the practice of the present invention include anesthetics, anticonvulsants, antidepressants, antidiabetic agents, antidotes, antiemetics, antihistamines, anti-infective agents, antineoplastics, antiparkisonian drugs, antirheumatic agents, antipsychotics, anxiolytics, appetite stimulants and suppressants, blood modifiers, cardiovascular agents, central nervous system stimulants, drugs for Alzheimer's disease management, drugs for cystic fibrosis management, diagnostics, dietary supplements, drugs for erectile dysfunction, gastrointestinal agents, hormones, drugs for the treatment of alcoholism, drugs for the treatment of addiction, immunosuppressives, mast cell stabilizers, migraine preparations, motion sickness products, drugs for multiple sclerosis management, muscle relaxants, nonsteroidal anti-inflammatories, opioids, other analgesics and stimulants, opthalmic preparations, osteoporosis preparations, prostaglandins, respiratory agents, sedatives and hypnotics, skin and mucous membrane agents, smoking cessation aids, Tourette's syndrome agents, urinary tract agents, and vertigo agents.

Examples of anesthetic agents include ketamine and lidocaine.

Examples of anticonvulsants include compounds from one of the following classes: GABA analogs, tiagabine, vigabatrin; barbiturates such as pentobarbital; benzodiazepines such as clonazepam; hydantoins such as phenytoin; phenyltriazines such as lamotrigine; miscellaneous anticonvulsants such as carbamazepine, topiramate, valproic acid, and zonisamide.

Examples of antidepressants include amitriptyline, amoxapine, benmoxine, butriptyline, clomipramine, desipramine, dosulepin, doxepin, imipramine, kitanserin, lofepramine, medifoxamine, mianserin, maprotoline, mirtazapine, nortriptyline, protriptyline, trimipramine, venlafaxine, viloxazine, citalopram, cotinine, duloxetine, fluoxetine, fluvoxamine, milnacipran, nisoxetine, paroxetine, reboxetine, sertraline, tianeptine, acetaphenazine, binedaline, brofaromine, cericlamine, olovoxamine, iproniazid, isocarboxazid, moclobemide, phenyhydrazine, pheneizine, selegiline, sibutramine, tranylcypromine, ademetionine, adrafmil, amesergide, amisulpride, amperozide, benactyzine, bupropion, caroxazone, gepirone, idazoxan, metralindole, milnacipran, minaprine, nefazodone, nomifensine, ritanserin, roxindole, Sadenosylmethionine, escitalopran, tofenacin, trazodone, tryptophan, and zalospirone.

Examples of antidiabetic agents include pioglitazone, rosiglitazone, and troglitazone.

Examples of antidotes include edrophonium chloride, flumazenil, deferoxamine, nalmefene, naloxone, and naltrexone.

Examples of antiemetics include alizapride, azasetron, benzquinamide, bromopride, buclizine, chlorpromazine, cinnarizine, clebopride, cyclizine, diphenhydramine, diphenidol, dolasetron, droperidol, granisetron, hyoscine, lorazepam, dronabinol, metoclopramide, metopimazine, ondansetron, perphenazine, promethazine, prochlorperazine, scopolamine, triethylperazine, trifluoperazine, triflupromazine, trimethobenzamide, tropisetron, domperidone, and palonosetron.

Examples of antihistamines include astemizole, azatadine, brompheniramine, carbinoxamine, cetrizine, chlorpheniramine, cinnarizine, clemastine, cyproheptadine, dexmedetomidine, diphenhydramine, doxylamine, fexofenadine, hydroxyzine, loratidine, promethazine, pyrilamine, and terfenidine.

Examples of anti-infective agents include compounds selected from one of the following classes: antivirals such as efavirenz; AIDS adjunct agents such as dapsone; aminoglycosides such as tobramycin; antifungals such as fluconazole; antimalarial agents such as quinine; antituberculosis agents such as ethambutol; P-lactams such as cefmetazole, cefazolin, cephalexin, cefoperazone, cefoxitin, cephacetrile, cephaloglycin, cephaloridine; cephalosporins, such as cephalosporin C, cephalothin; cephamycins such as cephamycin A, cephamycin B, and cephamycin C, cephapirin, cephradine; leprostatics such as clofazimine; penicillins such as ampicillin, amoxicillin, hetacillin, carfecillin, carindacillin, carbenicillin, amylpenicillin, azidocillin, benzylpenicillin, clometocillin, eloxacillin, cyclacillin, methicillin, nafcillin, 2-pentenylpenicillin, penicillin N, penicillin O, penicillin S, penicillin V, dicloxacillin; diphenicillin; heptylpenicillin; and metampicillin; quinolones such as eiprofloxacin, clinafloxacin, difloxacin, grepafloxacin, norfioxacin, ofloxacine, temafloxacin; tetracyclines such as doxycycline and oxytetracycline; miscellaneous anti-infectives such as linezolide, trimethoprim and sulfamethoxazole.

Examples of anti-neoplastic agents include droloxifene, tamoxifen, and toremifene.

Examples of anti-parkisonian drugs include amantadine, baclofen, biperiden, benztropine, orphenadrine, procyclidine, trihexyphenidyl, levodopa, carbidopa, andropinirole, apomorphine, benserazide, bromocriptine, budipine, cabergoline, eliprodil, eptastigmine, ergoline, galanthamine, lazabemide, lisuride, mazindol, memantine, mofegiline, pergolide, piribedil, pramipexole, propentofylline, rasagiline, remacemide, ropinerole, selegiline, spheramine, terguride, entacapone, and tolcapone.

Examples of anti-rheumatic agents include diclofenac, hydroxychloroquine, and methotrexate.

Examples of antipsychotics include acetophenazine, alizapride, amisuipride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, loxapine, melperone, mesoridazine, metofbnazate, molindrone, olanzapine, penfluridol, pericyazine, perphenazine, pimozide, pipamerone, piperacetazine, pipotiazine, prochiorperazine, promazine, quetiapine, remoxipride, risperidone, sertindole, spiperone, sulpiride, thioridazine, thiothixene, trifluperidol, triflupromazine, trifluoperazine, ziprasidone, zotepine, and zuclopenthixol.

Examples of anxiolytics include alprazolam, bromazepam, oxazepam, buspirone, hydroxyzine, mecloqualone, medetomidine, metomidate, adinazolam, chlordiazepoxide, clobenzepam, flurazepam, lorazepam, loprazolam, midazolam, alpidem, alseroxlon, amphenidone, azacyclonol, bromisovalum, captodiarnine, capuride, carbcloral, carbromal, chloral betaine, eneiprazine, flesinoxan, ipsapiraone, lesopitron, loxapine, methaqualone, methprylon, propanolol, tandospirone, trazadone, zopiclone, and zolpidem.

An example of an appetite stimulant is dronabinol.

Examples of appetite suppressants include fenfluramine, phentermine, and sibutramine.

Examples of blood modifiers include cilostazol and dipyridamol.

Examples of cardiovascular agents include benazepril, captopril, enalapril, quinapril, ramipril, doxazosin, prazosin, clonidine, labetolol, candesartan, irbesartan, losartan, telmisartan, valsartan, disopyramide, flecanide, mexiletine, procainaniide, propafenone, quinidine, tocainide, amiodarone, dofetilide, ibutilide, adenosine, gemfibrozil, lovastatin, acebutalol, atenolol, bisoprolol, esmolol, metoprolol, nadolol, pindolol, propranolol, sotalol, diltiazem, nifedipine, verapamil, spironolactone, bumetanide, ethacrynic acid, furosemide, torsemide, amiloride, triamterene, and metolazone.

Examples of central nervous system stimulants include amphetamine, brucine, caffeine, dexfenfluramine, dextroamphetamine, ephedrine, fenfluramine, mazindol, methyphenidate, pemoline, phentermine, sibutramine, and modafinil.

Examples of drugs for Alzheimer's disease management include donepezil, galanthamine, and tacrin.

Examples of drugs for cystic fibrosis management include ciprofloxacin, 3-isobutyl-1-methylxanthine, XAC and analogues, 4-phenylbutyric acid, genistein and analogous isoflavones, and milrinone.

Examples of diagnostic agents include adenosine and and aminohippuric acid.

Examples of dietary supplements include melatonin and vitamin-E.

Examples of drugs for erectile dysfunction include tadalafil, sildenafil, vardenafil, apomorphine, apomorphine diacetate, phentolamine, and yohimbine.

Examples of gastrointestinal agents include loperamide, atropine, hyoscyamine, famotidine, lansoprazole, omeprazole, and rebeprazole.

Examples of hormones include: testosterone, estradiol, and cortisone.

Examples of drugs for the treatment of alcoholism include naloxone, naltrexone, and disulfiram.

An example of a drug for the treatment of addiction is buprenorphine.

Examples of immunosupressives include mycophenolic acid, cyclosporin, azathioprine, tacrolimus, and rapamycin.

Examples of mast cell stabilizers include cromolyn, pemirolast, and nedocromil.

Examples of drugs for migraine headache include almotriptan, alperopride, codeine, dihydroergotamine, ergotamine, eletriptan, frovatriptan, isometheptene, lidocaine, lisuride, metoclopramide, naratriptan, oxycodone, propoxyphene, rizatriptan, sumatriptan, tolfenamic acid, zolmitriptan, amitriptyline, atenolol, clonidine, cyproheptadine, diltiazem, doxepin, fluoxetine, lisinopril, methysergide, metoprolol, nadolol, nortriptyline, paroxetine, pizotifen, pizotyline, propanolol, protriptyline, sertraline, timolol, and verapamil.

Examples of motion sickness products include diphenhydramine, promethazine, and scopolamine.

Examples of drugs for multiple sclerosis management include bencyclane, methylprednisolone, mitoxantrone, and prednisolone.

Examples of muscle relaxants include baclofen, chlorzoxazone, cyclobenzaprine, methocarbamol, orphenadrine, quinine, and tizanidine.

Examples of nonsteroidal anti-inflammatory drugs include aceclofenac, acetaminophen, alminoprofen, amfenac, aminopropylon, amixetrine, aspirin, benoxaprofen, bromfenac, bufexamac, carprofen, celecoxib, choline, salicylate, cinchophen, cinmetacin, clopriac, clometacin, diclofenac, diflunisal, etodolac, fenoprofen, flurbiprofen, ibuprofen, indomethacin, indoprofen, ketoprofen, ketorolac, mazipredone, meclofenamate, nabumetone, naproxen, parecoxib, piroxicam, pirprofen, rofecoxib, sulindac, tolfenamate, tolmetin, and valdecoxib.

Examples of opioid drugs include alfentanil, allylprodine, alphaprodine, anileridine, benzylmorphine, bezitramide, buprenorphine, butorphanol, carbiphene, cipramadol, clonitazene, codeine, dextromoramide, dextropropoxyphene, diamorphine, dthydrocodeine, diphenoxylate, dipipanone, fentanyl, hydromorphonc, L-alpha acetyl methadol, lofentanil, levorphanol, meperidine, methadone, meptazinol, metopon, morphine, nalbuphine, nalorphine, oxycodone, papaveretum, pethidine, pentazocine, phenazocine, remifentanil, sufentanil, and tramadol.

Examples of other analgesic drugs include apazone, benzpiperylon, benzydramine, caffeine, clonixin, ethobeptazine, flupirtine, nefopam, orphenadrine, propacetamol, and propoxyphene.

Examples of opthalmic preparation drugs include ketotifen and betaxolol.

Examples of osteoporosis preparation drugs include alendronate, estradiol, estropitate, risedronate, and raloxifene.

Examples of prostaglandin drugs include epoprostanol, dinoprostone, misoprostol, and alprostadil.

Examples of respiratory agents include albuterol, ephedrine, epinephrine, fomoterol, metaproterenol, terbutaline, budesonide, ciclesonide, dexamethasone, flunisolide, fluticasone propionate, triamcinolone acetonide, ipratropium bromide, pseudoephedrine, theophylline, montelukast, zafirlukast, ambrisentan, bosentan, enrasentan, sitaxsentan, tezosentan, iloprost, treprostinil, and pirfenidone.

Examples of sedative and hypnotic drugs include butalbital, chlordiazepoxide, diazepam, estazolam, flunitrazepam, flurazepam, lorazepam, midazolam, temazepam, triazolam, zaleplon, zolpidem, and zopiclone.

Examples of skin and mucous membrane agents include isotretinoin, bergapten, and methoxsalen.

Examples of smoking cessation aids include nicotine and varenicline.

An example of a drug for the treatment of Tourette's is pimozide.

Examples of urinary tract agents include tolteridine, darifenicin, propantheline bromide, and oxybutynin.

Examples of vertigo agents include betahistine and indolizine.

In certain embodiments, a drug can further comprise substances to enhance, modulate, and/or control release, aerosol formation, intrapulmonary delivery, therapeutic efficacy, therapeutic potency, and/or stability of the drug. For example, to enhance therapeutic efficacy, a drug can be co-administered with one or more active agents to increase the absorption or diffusion of the first drug through the pulmonary alveoli, or to inhibit degradation of the drug in the systemic circulation. In certain embodiments, a drug can be co-administered with active agents having pharmacological effects that enhance the therapeutic efficacy of the drug. In certain embodiments, a drug can comprise compounds that can be used in the treatment of one or more diseases, conditions, or disorders. In certain embodiments, a drug can comprise more than one compound for treating a disease, condition, or disorder, or for treating more than one disease, condition, or disorder.

A film of drug can be applied to the substrate (or component 602 of FIG. 6) by any appropriate method, depending on such factors as the physical properties of the specific drug and the thickness of the film, among others. In certain embodiments, methods of applying a drug to the exterior substrate surface include, but are not limited to, brushing, dip coating, spray coating, screen printing, roller coating, inkjet printing, vapor-phase deposition, and spin coating. In certain embodiments, the drug can be prepared as a solution comprising at least one solvent and applied to the exterior surface. In certain embodiments, a solvent can comprise a volatile solvent such as, for example, but without limitation, acetone or isopropanol. In certain embodiments, the drug can be applied to the exterior surface of the substrate as a melt. In certain embodiments, the drug can be applied to a support having a release coating and transferred to a substrate from the support. For drugs that are liquid at room temperature, thickening agents can be admixed with the drug to produce a viscous composition comprising the drug that can be applied to the exterior substrate surface by any appropriate method, including those listed above. In certain embodiments, a film of compound can be formed during a single application, or can be formed during repeated applications, to increase the final thickness of the film. In certain embodiments, the final thickness of a film of drug disposed on the exterior substrate surface can be less than 50 μm; in certain embodiments, less than 20 μm; in certain embodiments, less than 10 μm; in certain embodiments, within the range of 0.1 μm to 10 μm.

In certain embodiments, the film can comprise a therapeutically effective amount of at least one drug. “Therapeutically effective amount” refers to an amount sufficient to affect treatment when administered to a patient or user in need of treatment. Treating or treatment of a disease, condition, or disorder refers to arresting or ameliorating; reducing the risk of acquiring; reducing the development of, or at least one of the clinical symptoms of; or reducing the risk of developing, or at least one of the clinical symptoms of, a disease, condition, or disorder. Treating or treatment also refers to inhibiting the disease, condition, or disorder, either physically (e.g., stabilization of a discernible symptom), physiologically (e.g., stabilization of a physical parameter), or both, and inhibiting at least one physical parameter that may not be discernible to the patient. Further, treating or treatment refers to delaying the onset of the disease, condition, or disorder, or at least symptoms thereof, in a patient which may be exposed to or predisposed to a disease, condition, or disorder, even though that patient does not yet experience or display symptoms of the disease, condition, or disorder.

In certain embodiments, the film can comprise one or more pharmaceutically acceptable carriers, adjuvants, and/or excipients. “Pharmaceutically acceptable” refers to approved or approvable by a regulatory agency of the Federal or a state government or listed in the U.S. Pharmacopoeia or other generally recognized pharmacopoeia for use in animals, and more particularly, in humans.

The drug can be disposed on the substrate in any appropriate form such as a solid, viscous liquid, liquid, crystalline solid, or powder. In certain embodiments, the film of drug can be crystallized after disposition on the substrate.

In one aspect, the second surface of the above-described substrate may have a plurality of regions, such that different drugs can be disposed on different regions, thereby facilitating delivery of different drugs from the same device and/or the delivery of drugs in a specified sequence.

The above-described drug supply units facilitate producing an aerosol of a drug. This can be readily accomplished by initiating an exothermic reaction of the fuel material of the above-described drug supply unit, thereby vaporizing the drug. Thus, a drug supply unit according to the present invention is configured such that the fuel material heats a portion of the exterior surface of the substrate to a temperature sufficient to thermally vaporize the drug, in certain embodiments within 3 seconds following ignition of the fuel material, in other embodiments within 1 second following ignition of the fuel material, in other embodiments within 800 milliseconds following ignition of the fuel material, in other embodiments within 500 milliseconds following ignition of the fuel material, and in other embodiments within 250 milliseconds following ignition of the fuel material.

In certain embodiments, a drug supply unit can generate an aerosol comprising a drug that can be inhaled directly by a user and/or can be mixed with a delivery vehicle, such as a gas, to produce a stream for delivery (for example, via a spray nozzle) to a topical site for a variety of treatment regimens, including acute or chronic treatment of a skin condition, administration of a drug to an incision site during surgery, or to an open wound.

In certain embodiments, rapid vaporization of a drug film can occur with minimal thermal decomposition of the drug. For example, in certain embodiments, less than 10% of the drug is decomposed during thermal vaporization, and in certain embodiments, less than 5% of the drug is decomposed during thermal vaporization. In certain embodiments, a drug can undergo a phase transition to a liquid state and then to a gaseous state, or can sublime (i.e., pass directly from a solid state to a gaseous state).

In certain embodiments, drug aerosol purities and particle sizes upon thin drug film vaporization can be modulated by controlling the propagation speed of the heating wavefront. The chemical kinetics associated with thermal vaporization of a thin layer of drug play a key role in determining drug particle characteristics. Different propagation speeds can alter the amount of drug vaporized at a given time and thereby control the drug purities and/or particle sizes.

### EXAMPLES

### Example One

### Preparation of Laponite® Gel

Deionized water (170 g) was weighed into a container, then stirred in a general purpose mixer (VWR International, West Chester, Pa.) at speed 3. Thirty grams (30 g) of Laponite RDS additive (Southern Clay Products, Gonzales, Tex.) was added to the stirring water. Stirring was continued at speed 3 for 25 minutes, resulting in the formation of a clear gel, which was subsequently transferred to a syringe reservoir.

### Example Two

### Preparation of Chemical Reactant Formulations

The chemical reactant formulations listed in Table One, below, were prepared according to the method described below. Iron oxide (Fe₂O₃, ca. 0.8 μm) was obtained from Elementis (East St. Louis, Ill.); manganese dioxide (MnO₂, <45 μm) was obtained from Alfa Aesar (Ward Hill, Mass.) and zirconium (Zr, AB grade, ca. 3.0 μm) from Chemetall (Frankfurt, Germany).

TABLE ONE
Preparation of Chemical Reactant Formulations*

| Component | Reactant A | Reactant B | Reactant C |
|---|---|---|---|
| Zr | 72 g | 56.25 g | 45.36 g |
| | (0.82 mol) | (0.60 mol) | (0.48 mol) |
| Fe₂O₃ | 28 g | 18.75 g | 17.64 g |
| | (0.18 mol) | (0.12 mol) | (0.11 mol) |
| MnO₂ | — | 25 g | 37 g |
| | | (0.28 mol) | (0.41 mol) |

*Masses listed are for a 100 g batch.

The compositions listed in Table One, above, were mixed with an aqueous solution of Laponite (prepared as described in Example One, above) using a Thinky® mixer (Tokyo, Japan). After thorough mixing, the resulting slurries were transferred to syringe reservoirs and allowed to sit for at least 6 hours before coating the desired coating density onto stainless steel foil substrates (obtained from Ulbrich Stainless Steels & Special Metals, Inc., North Haven, Conn.) using an automated tip dispenser (Intelligent Actuators, Torrance, Calif.). The correlation between stainless steel foil substrate temperature and reactant coating density for the Reactants A, B, and C (prepared as described above) is illustrated in FIG. 2, which is a graph 200 showing peak substrate surface temperatures 202 achieved by the foil substrates as a function of reactant coating density 204 (in mg/cm²). Curves 206, 208, and 210 represent Reactants A, B, and C, respectively.

As can be seen from FIG. 2, for Reactant B, an increase (i.e., slope) in peak substrate surface temperature of 14.5° C. occurs for each 1 mg/cm² increase in reactant coating density, while Reactant A, which lacks MnO₂, exhibits a larger peak substrate surface temperature increase of 20.1° C. for each 1 mg/cm² increase in reactant coating density. Reactant C exhibits a heat-buffering exothermic reaction, i.e., the peak substrate surface temperature for Reactant C exhibits a much smaller increase with increased reactant coating density than either Reactant B or Reactant C. The peak substrate surface temperature levels off quickly, and does not continue to rise with increasing reactant coating density within the buffering region. The variation in slopes shown in FIG. 2 is reflective of differential heat output and chemistries involved. The data shown in FIG. 2 indicate that different peak substrate surface temperatures can be attained by using either distinct formulation ratios (Reactants A, B, and C) or identical formulations with varying coating densities. Although the peak substrate surface temperature generally increases with higher coating density, we made an interesting discovery that certain reactant formulations (such as Reactant C) exhibit a non-linear correlation leading to sustained/controlled heat release. For example, the reaction end-products of Reactant C seem to undergo a phase transition that leads to a peak substrate surface temperature of 430° C. regardless of the increments in coating density (above a threshold coating density of 11.0 mg/cm²). X-ray powder diffraction of Reactant C's end-products indicates the presence of an alloy of Fe and Mn in addition to ZrO₂, which could be responsible for the heat buffering activity. Further research on this fascinating property may shed light on novel self-propagating chemical reactions and controlled heat-releasing systems that are of fundamental scientific importance.

### Example Three

### Thermal Profiles of Heated Substrates

The exothermic reactant formulations based on different mole ratios of Zr, Fe₂O₃ (ΔH_{f}=−822.2 kJ/mol), and MnO₂ (ΔH_{f}=−519.7 kg/mol) were used to generate varying amounts of thermal energy to alter the surface temperatures (300-450° C.) of the steel foil substrate and the reaction propagation speeds (0.04-20.0 m/s). Thermal profiles of the heated substrates were analyzed using an infrared thermal imaging camera (FLIR, North Billerica, Mass.).

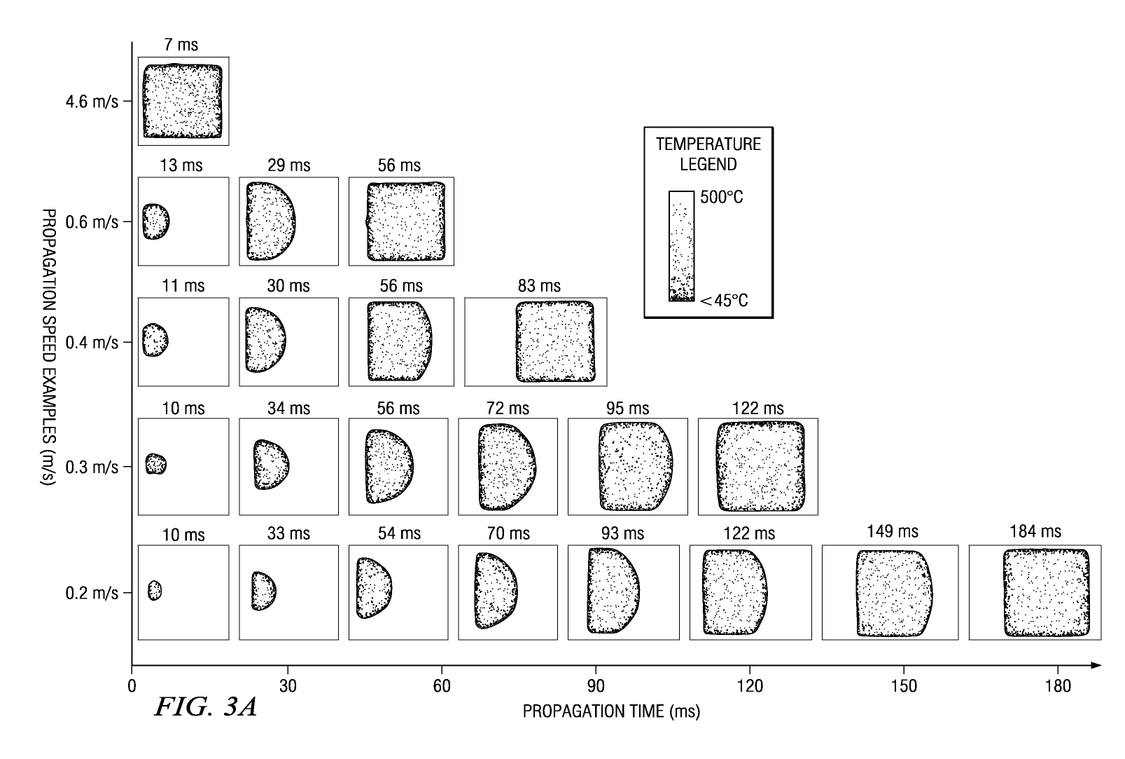

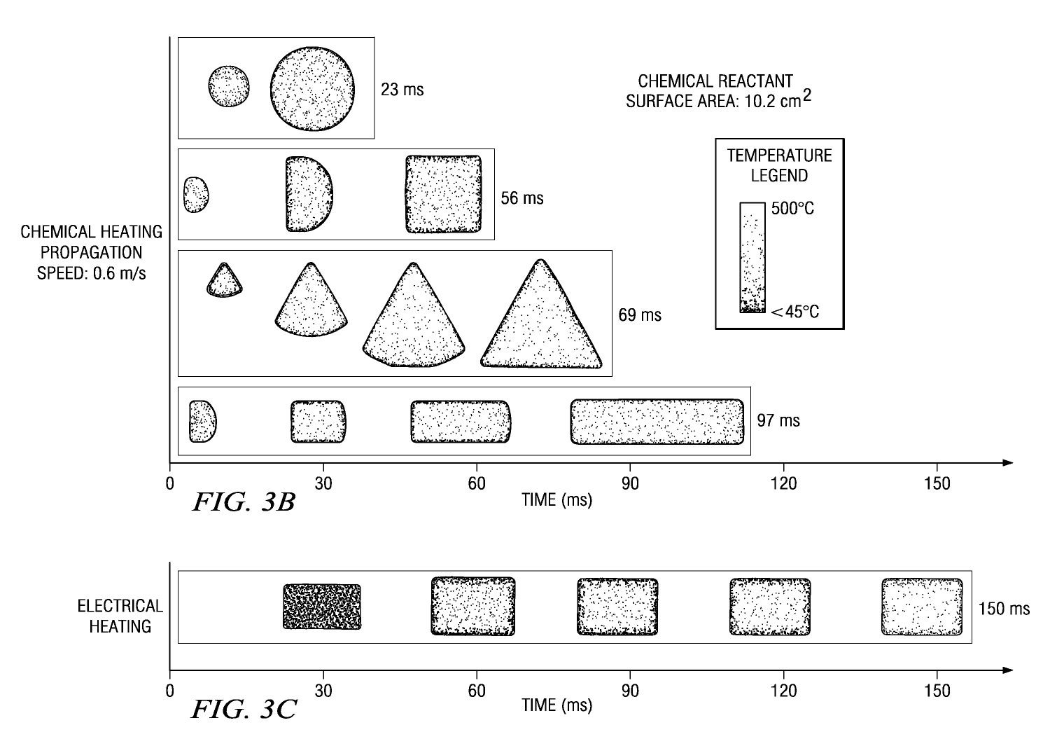

FIGS. 3A-3C are schematic representations of infrared thermal images of stainless steel foil substrates heated using self-propagating chemical reactions and electricity. FIG. 3A shows infrared thermal images of stainless steel foil substrates coated with different reactant formulation ratios yielding various propagation speeds (0.2, 0.3, 0.4, 0.6, and 4.6 m/s), and the reactants were coated onto the foils as square shapes (10.2 cm²). FIG. 3B shows infrared thermal images of stainless steel foil substrates, where a single reactant formulation was coated onto foil substrates as different shapes (i.e., circle, square, triangle, and rectangle, from top to bottom) with identical surface areas (10.2 cm²) and propagation speeds (0.6 m/s), but where the reaction was initiated from different points on the reactant coating. FIG. 3C infrared thermal images of a stainless steel foil substrate (2.25 cm length×1.5 cm width×0.0127 cm thickness) resistively heated using a 1 Farad capacitor to highlight the differences between exothermic and electric heating methods drug vaporization concentrations with time. The peak substrate surface temperature in all of the above cases (i.e., FIGS. 3A, 3B, and 3C) was approximately 400° C.

Without intending to be bound by theory, we believe that “latent heat” and phase transformation properties of the products formed at the end of the exothermic reaction are responsible for both heat buffering and rapid heating properties. In principle, if an end product of any new exothermic reactant formulation exhibits desirable latent heat/phase transformation properties, it might function as a heat buffer. Also, the end products of the exothermic reactant formulations with heat buffering properties may have high thermal energy storage capacity. Therefore, we believe that our discovery has broad applicability and may shed light on a whole new series of materials with interesting heat storage and/or heat transfer properties (e.g., thermal batteries).

### Example Four

### Propagation Speed Can Affect Drug Particle Size

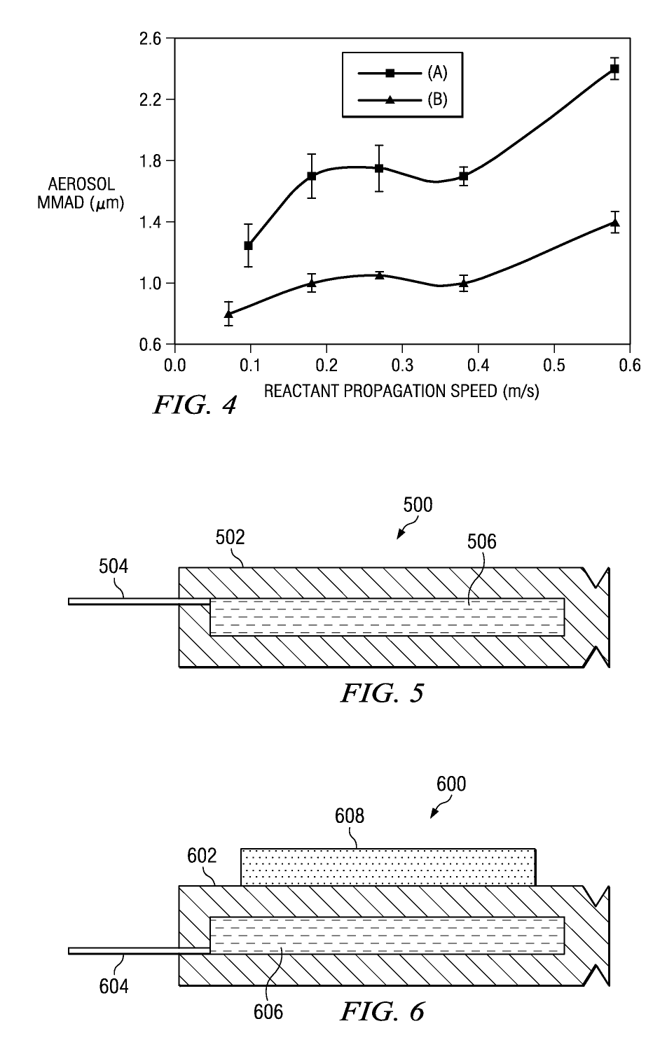

Fast propagating reactant formulations generate larger particle sizes, whereas slow propagating wavefronts yield smaller particle sizes. Different propagation speeds alter the amount of drug vaporized at a given time and thereby affect drug particle sizes. FIG. 4 shows particle size data of thin drug films vaporized at 380° C. with 6.4 m/s air velocity and 28.3 L/min airflow, in which the particle sizes were measured with an Andersen cascade impactor (n=3 per data point). A 5.0 mg dose of prochlorperazine (Data 4A, drug coating density: 1.1 mg/cm²) changes its particle size from 2.4 to 1.4 μm (mass median aerodynamic diameter, MMAD) when the propagation speed is decreased by six-fold. A similar change in propagation speed for a 0.75 mg dose of bumetanide (Data 4B, drug coating density: 0.10 mg/cm²) decreases the particle sizes also, from 1.4 to 0.8 μm (MMAD). We observed similar trends for different drug coating densities. These results clearly demonstrate the advantages of chemical heating methods to precisely fine-tune drug particle sizes to the required range for a given drug dosage and drug coating density.

It is to be understood that, while the invention has been described in conjunction with the preferred specific embodiments thereof, that the description above as well as the examples that follow are intended to illustrate and not limit the scope of the invention. The practice of the present invention will employ, unless otherwise indicated, conventional techniques of chemistry, manufacturing and engineering, and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

Various embodiments of the disclosure could also include permutations of the various elements recited in the claims as if each dependent claim was multiple dependent claim incorporating the limitations of each of the preceding dependent claims as well as the independent claims. Such permutations are expressly within the scope of this disclosure.

That which is claimed is:

1. A reactant formulation capable of undergoing an exothermic chemical reaction, wherein the reactant formulation comprises a metal reducing agent, a metal-containing oxidizing agent, and manganese oxide, wherein the manganese oxide is present at a concentration sufficient to control a temperature profile of the reactant formulation.

2. The reactant formulation of claim 1, wherein the manganese oxide is present at a concentration within the range of 2 to 60 percent by weight of the reactant formulation.

3. The reactant formulation according to claim 2, wherein the manganese oxide is present at a concentration within the range of 8 to 40 percent by weight of the reactant formulation.

4. The reactant formulation according to claim 3, wherein the manganese oxide is present at a concentration within the range of 20 to 40 percent by weight of the reactant formulation.

5. The reactant formulation according to claim 1, wherein the manganese oxide is manganese dioxide.

6. The reactant formulation according to claim 1, wherein the metal reducing agent is selected from the group consisting of: zirconium, molybdenum, magnesium, calcium, strontium, barium, boron, titanium, vanadium, niobium, tantalum, chromium, tungsten, manganese, iron, cobalt, nickel, copper, zinc, cadmium, tin, antimony, bismuth, aluminum, and silicon.

7. The reactant formulation according to claim 1, wherein the metal reducing agent is zirconium.

8. The reactant formulation according to claim 1, wherein the zirconium is present at a concentration within the range of 30 to 90 percent by weight of the reactant formulation.

9. The reactant formulation according to claim 8, wherein the zirconium is present at a concentration within the range of 40 to 80 percent by weight of the reactant formulation.

10. The reactant formulation according to claim 9, wherein the zirconium is present at a concentration within the range of 40 to 70 percent by weight of the reactant formulation.

11. The reactant formulation according to claim 1, wherein the metal-containing oxidizing agent is selected from the group consisting of: transition metal oxides, lanthanide metal oxides, and mixed metal oxides.

12. The reactant formulation according to claim 11, wherein the metal-containing oxidizing agent is a transition metal oxide selected from the group consisting of oxides of: iron, copper, cobalt, molybdenum, vanadium, chromium, manganese, silver, tungsten, magnesium, and niobium.

13. The reactant formulation according to claim 12, wherein the metal-containing oxidizing agent is selected from the group consisting of: Fe₂O₃, CuO, Co₃O₄, Co₂O₃, and MoO₃.

14. The reactant formulation according to claim 13, wherein the metal-containing oxidizing agent is iron oxide.

15. The reactant formulation according to claim 14, wherein the iron oxide is present at a concentration within the range of 5 to 40 percent by weight of the reactant formulation.

16. The reactant formulation according to claim 15, wherein the iron oxide is present at a concentration within the range of 10 to 30 percent by weight of the reactant formulation.

17. The reactant formulation according to claim 16, wherein the iron oxide is present at a concentration within the range of 15 to 30 percent by weight of the reactant formulation.

18. The reactant formulation according to claim 1, wherein the reactant formulation further comprises a binding agent.

19. The reactant formulation according to claim 18, wherein the binding agent is selected from the group consisting of: clays, metal silicates, phosphate-containing materials, alkoxides, metal oxides, inorganic polyanions, inorganic polycations, inorganic sol-gel materials, synthetic ion exchange resins, zeolites, and diatomaceous earth.

20. The reactant formulation according to claim 19, wherein the binding agent is Laponite®.

21. An article useful for vaporization of a vaporizable compound, wherein the article comprises:
a substrate having a first surface and a second surface, wherein at least a portion of the first surface is coated with a reactant formulation capable of undergoing an exothermic chemical reaction, wherein the reactant formulation comprises a metal reducing agent, a metal oxidizing agent, and manganese oxide, wherein the manganese oxide is present at a concentration sufficient to control a temperature profile of the reactant formulation, and
wherein at least a portion of the second surface of the substrate is coated with a vaporizable compound.

22. The article of claim 21, wherein the reactant formulation is present at a concentration wherein the range of 2 to 60 percent by weight of the reactant formulation.

23. The article according to claim 21, wherein the vaporizable compound comprises a drug.

24. A method of controlling a temperature profile of a chemical reactant formulation, wherein the method comprises combining the chemical reactant formulation with manganese oxide, wherein the manganese oxide present at a concentration sufficient to control a temperature profile of the relevant formulation upon indication of the chemical reactant formulation.

25. The method according to claim 24, wherein the concentration of manganese oxide within the range of 2 to 60 percent by weight of the chemical reactant formulation.

26. The method according to claim 24, wherein the concentration of manganese oxide within the chemical reactant formulation controls the rate of heating and/or heat propagation of the initiated chemical reactant formulation.

27. The method according to claim 24, wherein the concentration of manganese oxide within the chemical reactant formulation controls the maximum temperature that can be attained by the initiated chemical reactant formulation.

28. The method according to claim 24, wherein the concentration of manganese oxide within the chemical reactant formulation controls the timeframe over which the initiated chemical reactant formulation is able to maintain a desired temperature.

29. A method of improving the uniformity of heating upon ignition of a chemical reactant formulation, wherein the method comprises combining the chemical reactant formulation with manganese oxide, wherein the manganese oxide is present at a concentration within the range of 2 to 60 percent by weight of the chemical reactant formulation.

30. The method according to claim 29, wherein uniformity of heating upon initiation of the chemical reactant formulation is controlled by varying the percentage of manganese oxide within the chemical reactant formulation.

31. A method of improving the adhesion of a chemical reactant formulation to a substrate upon initiation of the chemical reactant formulation, wherein the method comprises combining the chemical reactant formulation with manganese oxide, wherein the manganese oxide is present at a concentration within the range of 2 to 60 percent by weight of the chemical reactant formulation.

32. The method according to claim 31, wherein the degree to which the adhesion of the chemical reactant formulation to substrate is enhanced is varied by varying the percentage of manganese oxide within the chemical reactant formulation.

33. A method of modulating the maximum temperature achieved upon initiation of a chemical reactant formulation, wherein the method comprises combining the chemical reactant formulation with manganese oxide, wherein the manganese oxide is present at a concentration within the range of 2 to 60 percent by weight of the chemical reactant formulation.

34. A heating unit comprising:
a substrate having a first surface and a second surface; and
a reactant formulation capable of undergoing an exothermic chemical reaction disposed upon at least a portion of the first surface of the substrate, wherein the chemical reactant formulation comprises a metal reducing agent, a metal-containing oxidizing agent, and manganese oxide, wherein the manganese oxide is present at a concentration sufficient to control a temperature profile of the reactant formulation.

35. The heating unit of claim 34 wherein the manganese oxide is present at a concentration within the range of 2 to 60 percent by weight of the chemical reactant formulation.

36. The heating unit according to claim 34, further comprising at least one vaporizable compound disposed upon at least a portion of the second surface of the substrate.

37. A heating unit according to claim 34, wherein the vaporizable compound comprises a drug.
